(19) United States
(12) Patent Application Publication
(10) Pub. No.: US 2020/0188326 A1
(43) Pub. Date: Jun. 18, 2020

(54) COMPOSITIONS AND METHODS OF TREATMENT FOR MVID AND RELATED DISEASES

(71) Applicant: Vanessa Research, Inc., Hamden, CT (US)

(72) Inventors: Dmitry KRAVTSOV, Milford, CT (US); Norman GRAY, Hamden, CT (US)

(73) Assignee: Vanessa Research, Inc., Hamden, CT (US)

(21) Appl. No.: 16/545,864

(22) Filed: Aug. 20, 2019

Related U.S. Application Data
(63) Continuation of application No. 15/521,715, filed on Jun. 22, 2018, now abandoned.
(60) Provisional application No. 62/387,153, filed on Dec. 23, 2015.

(51) Int. Cl. A61K 31/07; A61K 36/87; A61K 31/05; A61K 31/203; A61K 33/30; A61K 36/45; A61K 36/47; A61K 36/736; A61K 36/738; A61K 36/185; A61K 31/11; A61P 1/12; A61K 45/06

(52) U.S. Cl. CPC A61K 31/07; A61K 2300/00; A61K 31/05; A61K 31/203; A61K 33/30; A61K 36/45; A61K 36/47; A61K 36/736; A61K 36/738; A61K 36/185; A61K 31/11; A61P 1/12; A61K 45/06; Y02A 50/471; A61K 36/87

(57) ABSTRACT

A composition, comprising: a. a retinoid; b. a polyphenol; and c. a zinc salt, calcium salt, selenium salt, bismuth salt, or a combination thereof.

### RELATED APPLICATIONS

This application claims the benefit of U.S. Provisional Application No. 62/387,153, filed Dec. 23, 2015, the entire contents of which are hereby incorporated by reference.

### BACKGROUND

Microvillus Inclusion Disease (MVID) is a rare autosomal recessive disorder that presents in the first hours and days of life with intractable deadly diarrhea often after the first feeding.

Affected children produce volumes of watery stool often exceeding that seen in cholera. The disease clusters in infants of Saudi Arabian, Turkish and Navajo Indian descent, and in consanguineous families and appears as two variants, early and late onset, of which late onset can present itself within the first month of life and usually is less severe. Total Parenteral Nutrition (“TPN”) is indicated early in the course of the disease, which is fatal if left untreated. The pathognomonic feature of the disease is the presence of microvillus-containing inclusions within enterocytes, microvillus atrophy and prominent blunting of the small intestinal villi. There is no known organ involvement outside the gastrointestinal tract.

Research over the past 15 years has provided significant insights into the molecular mechanisms of MVID, including the discovery of the cause of the “early onset” disease—mutations in the Myosin Vb (Myo5b) gene by Muller et al (Muller, 2008) and “late onset” variant disease—mutations in the Syntaxin 3 (Syn3) gene by Wiegerinck et al (2014). The “early onset” variant is reported more frequently and inevitably fatal in the first years of life, unless a patient is treated surgically with total intestinal transplantation. In the latter case, the life expectancy is still very short with most patients dying before the age of 20.

Disruption of the cellular polarity was declared an emerging mechanism for the pathological findings in MVID as early as 1994 (Fish and Morris, 1994). The presence of intracellular vacuolar apical compartment (VAC) with microvilli was noted before in cells and tissues (Vega-Salas et al., 1988), (Achler et al., 1989; Carruthers et al., 1986; Ellinger and Pavelka, 1986). Current research efforts focus on the characterization of apical transport pathways connecting apical recycling endosomes (ARE) to the apical plasma membrane. Rab8 and Rab11 are uniformly accepted as small GTP-ases that are involved in the pathogenesis of MVID (Dhekne et al., 2014; Dong et al., 2012; Talmon et al., 2012, Knowles, 2014). Recently, aberrations in the phosphorylation of ezrin leading to the apical BB abnormalities were shown both in patient samples and Caco2 siRNA KD model (Dhekne et al., 2014).

Few attempts have been made to address the underlying pathogenesis of the secretory diarrhea in MVID. Davidson et al studied transport defects by marker perfusion of the proximal jejunum. Abnormal glucose absorption and a blunted response of Na⁺ absorption to actively transported nonelectrolytes was found in three infants whilst net secretion of Na⁺ and H2O was observed in the other two. Rhoads et al (Rhoads et al., 1991) analyzed proximal and distal intestinal tract outputs via a proximal jejunostomy placement in one patient showing that less fluid was lost from mouth to jejunum (60 ml/kgl—than from jejunum to anus (100 ml/kg/day). They also performed Na⁺, Cl⁻ and conductance analysis in the piece of jejunum excised during jejunostomy and reported that MVID epithelium displayed transmural conductance and unidirectional ion fluxes that were 30% of normal. With respect to both Na⁺ and Cl⁻, the excised MVID jejunum was in a net secretory state.

Phillips and Schmitz surveyed 23 cases of MVID in 1992 (Phillips and Schmitz, 1992) and reported that the presence of inclusions is characteristic of the mature enterocytes. Electron micrographs presented in their paper demonstrated that there are at least three different and distinctive appearances of the proximal small intestinal mucosa in the disease, and that not all cells in the disease completely lack microvilli; on the other hand, the cells possessing mature brush borders were hard to find.

Michail et al observed reduction in the expression levels of NHE2, NHE3 and SGLT1, but not NHE1 in the intestinal samples from MVID patients. The authors concluded that the patients with MVID have defects in apical, but not basolateral membrane transport systems, and that these defects are related to the pathogenesis of the disease (Michail et al., 1998). In 2000, Ameen and Salas executed a comparative analysis of protein distribution in a single intestinal tissue sample from a child diagnosed with MVID. In this study, apical (CFTR, actin, NHE3, Sucrase-Isomaltase, alkaline phosphatase and cGK II) and basolateral (Na-K ATP-ase) protein localization patterns were examined. The authors demonstrated that a number of BBM proteins were mis-localized in MVID enterocytes and redistributed to the cytosol and MVIs. The effect was more pronounced in the mature villus enterocytes. Distribution of basolateral Na-K-ATPase was not affected (Ameen and Salas, 2000). This observation is likely case-specific, as redistribution of transferrin receptor was later noted in MVID tissues (Dong et al., 2012). In addition, some latter reports demonstrated mis-localization of Na-K ATP-ase (Thoeni et al., 2014b). Increased leakiness and permeability of MVID intestine to the macromolecular substances was noted (Bijlsma et al, 2000).

There is evidence that severity of the diarrhea in MVID can be explained by defective absorption of the electrolytes and smaller nutrients in the milieu of continuous Cl⁻ secretion that leads to the tremendous losses of Na⁺ and Cl⁻ in stool (Kravtsov et al, 2016). The reason for selective abolishing of the absorptive intestinal function is a defect in development of the intestinal epithelium. Normally, the tall columnar epithelial cells of the intestinal lining shift from predominantly secreting crypt phenotype to predominantly absorbing villus phenotype as they mature and move up the crypt-villus axis. Maturation is accompanied by changes in the protein expression patterns, i.e., as cells become older, the expression of early secretive proteins, such as CFTR, decreases, while expression of absorptive proteins, such as SGLT1, DRA and NHE3, increases. HIPPO pathway members YAP/TAZ are examples of proteins controlling this process in the intestine. Polarity and transport abnormalities due to the loss of Myo5b release MVID cells from under the HIPPO pathway control, and the cells never acquire the proper absorptive phenotype. This defect seems to be related to the proper phosphorylation of the nuclear YAP molecules, and is functional rather then constitutive (Kravtsov et al, 2016). As a result, immature cells lacking absorptive capabilities, but well equipped for secretion, are left in charge of the intestine in an MVID patient, leading to the massive secretory diarrhea. Severity of the diarrhea is a result of a combinational defect involving multiple proteins. It is well known in pediatric practice that reduction in the expression of DRA or NHE3 alone leads to diarrheal diseases known as Congenital Chloride and Congenital Sodium Diarrhea, accordingly. In MVID, both proteins are reduced, adversely affecting the course of the disease.

### BRIEF DESCRIPTION OF THE SEVERAL EMBODIMENTS

One embodiment provides a composition, comprising:
a. a retinoid;
b. a polyphenol; and
c. a zinc salt, calcium salt, selenium salt, bismuth salt, or a combination thereof.

One embodiment provides a method for treating a subject having or suspecting to have one or more of MVID, congenital chloride diarrhea, congenital sodium diarrhea, diarrhea associated with Cholera, traveler's diarrhea, infectious diarrhea, secretory diarrhea, diarrhea associated with enterocyte development abnormality, tufting enteropathy, bacterial diarrhea, viral diarrhea, diarrhea associated with rotavirus infection, or a combination thereof, comprising administering to said subject a composition, comprising:
a. a retinoid;
b. a polyphenol; and
c. a zinc salt, calcium salt, selenium salt, bismuth salt, or a combination thereof.

### DETAILED DESCRIPTION OF THE SEVERAL EMBODIMENTS

In some embodiments, the composition is suitable for administration to a subject having or suspecting to have one or more of MVID, congenital chloride diarrhea, congenital sodium diarrhea, diarrhea associated with Cholera, traveler's diarrhea, infectious diarrhea, secretory diarrhea, diarrhea associated with enterocyte development abnormality, tufting enteropathy, bacterial diarrhea, viral diarrhea, diarrhea associated with rotavirus infection, or a combination thereof.

The retinoid is not particularly limited. In some embodiments, the retinoid may be selected from one or more of carotenoid, vitamin A, oral vitamin A, systemic Vitamin A, retinol, retinal, tretinoin, retinoic acid, isotretinoin, alitretinoin, etretinate, acitretin, tazarotene, bexarotene, adapalene and pyranon-derived retinoids, retinol palmitate, retinol acetate, Seletinoid G, 3,7-dimethyl-9-(2,6,6,trimethyl-1-cyclohexen-1-yl)-2,4,6,8-natetraen-1-ol, 3-dehydroretinol, Accutane®, acitretin, adapalene, alitretinoin, all-trans retinoic acid. Altinac®, Amnesteem®, antixerophthalmic vitamin, Aquasol A®, Avita®, axerophtholum, beta-carotene, beta-carotene oleovitamin A, bexarotene, Differin®, etretinate, isotretinoin, Palmitate-A®, Renova®, Retin-A®, Retin-A Micro®, retinaldehyde (RAL), retinyl acetate, retinyl N-formyl aspartamate, retinyl palmitate, retinol, Solatene®, Soriatane®, SourceCF®, Targretin®, tazarotene, Tazorac®, Tegison®, topical retinoids, tretinoin, Vesabiod®, Vesanoid®, Vitamax®, vitamin A USP, vitamin A1, vitamina A, vitaminum A, or a combination thereof.

In some embodiments, the retinoid is a carotenoid.

In some embodiments, the retinoid is one or more of vitamin A, retinol, retinal, tretinoin, retinoic acid, isotretinoin, alitretinoin, etretinate, acitretin, tazarotene, bexarotene, adapalene and pyranon-derived retinoids, retinol palmitate, retinol acetate, or a combination thereof.

In some embodiments, the retinoid is vitamin A, retinol palmitate, retinol acetate, or a combination thereof.

Vitamin A is a key vitamin in the regulation of cellular maturation and differentiation through the Retinoid Acid Receptors (http://www.jaad.org/article/S0190-9622(86)70231-4/abstract?cc=y=). In the intestine it was shown to significantly (up to 4 fold) upregulate the expression of SLC26A3 exchanger, which absorbs Cl⁻ from the intestinal lumen (http://www.ncbi.nlm.nih.gov/pubmed/?term=atra+dra+dudeja). It is used in the treatment of certain leukemias, where it induces the maturation of de-differentiated tumor cells and in the therapy of skin disorders. Both oral and injectable forms are contemplated, including slow-release depot preparations. Vitamin A derivatives will speed up the maturation of cells and recover the lost absorption of Na⁺, Cl⁻, glucose and amino acids via induction of the biosynthesis of the absorptive protein machinery. Dosage info on some forms: (http://www.karger.com/Article/Abstract/250839). Non-limiting examples include carotenoids, Vitamin A and its derivatives, retinoids, where “retinoid” can be, but not limited to, any of the following: oral and systemic Vitamin A, carotenoids, retinol, retinal, tretinoin (retinoic acid, Retin-A), isotretinoin, alitretinoin, etretinate, acitretin, tazarotene, bexarotene, adapalene and pyranon-derived retinoids, such as Seletinoid G., all-trans retinoic acid (ATRA), and other forms described such as in U.S. Pat. No. 4,190,594 A (https://www.google.com/patents/U.S. Pat. No. 4,190,594) and all its esterified derivatives. (Related terms 3,7-Dimethyl-9-(2,6,6,trimethyl-1-cyclohexen-1-yl)-2,4,6,8-natetraen-1-ol, 3-dehydroretinol, Accutane®, acitretin, adapalene, alitretinoin, all-trans retinoic acid, Altinac®, Amnesteem®, antixerophthalmic vitamin, Aquasol A®, Avita®, axerophtholum, beta-carotene, beta-carotene oleovitamin A, bexarotene, carotenoids, Differin®, etretinate, isotretinoin, Palmitate-A®, Renova®, Retin-A®, Retin-A Micro®, retinaldehyde (RAL), retinyl acetate, retinyl N-formyl aspartamate, retinyl palmitate, retinoic acid, retinol, Solatene®, Soriatane®, SourceCF®, Targretin®, tazarotene, Tazorac®, Tegison®, topical retinoids, tretinoin, Vesabiod®, Vesanoid®, Vitamax®, vitamin A USP, vitamin A1, vitamina A, vitaminum A.

The polyphenol is not particularly limited. In some embodiments, the polyphenol is a synthetic or naturally occurring polyphenol rich in —OH groups and having antiproliferative properties and/or inhibitory activity against ion and water transport proteins.

In some embodiments, the polyphenol is one or more of plant-derived, botanical, anthocyanoside, anthocyanidin, proanthocyanidin, tannin, or a combination thereof.

In some embodiments, the polyphenol is one or more of Alisitol, crofelemer, blueberry (Vaccinium cyanococcus spp), bilberry, sloe (Prunus Spinosa spp.), chokeberry (Aronia melanocarpa spp.), grape (Cabbernet sauvignon spp.), grape pomace, black rose, blackcurrant (Ribes nigrum spp.), pecarin, Dragon's Blood (Croton spp.) dried form thereof, seed form thereof, juice thereof, powder form thereof, liquid form thereof, extract thereof, or combination thereof.

Alisitol may refer to Vaccinium cyanococcus spp. (or fruit or dried powder thereof), Aronia melanocarpa spp. (fruit or dried powder of thereof) and/or Prunus spinose spp. (of fruit or dried powder of thereof), as a stand-alone form or in various mixtures, extract, suspension, solution, and any combination thereof, with or without seeds.

In some embodiments, the polyphenol is one or more of gallic acid, hydrolysable gallic acid, non-hydrolyzable gallic acid, condensed gallic acid, phlorotannin, cyanidin, delphinidin, petunidin, pelargonidin, peonidin, malvidin, catechin, gallocatechin, epicatechin, epigallocatechin, quercetin, tannic acid, apigenin, penta-m-digalloyl glucose, tannin, condensed tannin, gallotannic acid, gallotannin, tanninium, Cesinex, Styphnasal N, Zyone, monomeric form thereof, polymer thereof having degree of polymerization of 1, 2 or more, glycosylated form thereof, salt thereof, ester thereof, or combination thereof.

The polyphenol may be a botanical, or plant-derived polyphenol, preferably rich in —OH. Dark colored berries naturally reach in polyphenols, in particular blueberry (Vaccinium cyanococcus spp), bilberry, sloe (Prunus Spinosa spp.), chokeberry (Aronia melanocarpa spp.), grape (Cabbernet sauvignon spp.) and grape pomace, black rose, blackcurrant (Ribes nigrum spp.) (Pecarin) as well as other dark-colored berries, and all forms of extracts and preparations deriving from it. Dried blueberries are used as anti-diarrheal in the northern Europe. They contain the anthocyanosides that affect the secretory ion channels in the intestine and reduce secretion. They will combat the secretory component of MVID diarrhea.

Examples of berries and suppliers include Raw Pure Blueberry Powder, available from Four Seasons Gourmet Foods (via Amazon.com); Blueberry Powder 100 g. Reference: PFBLU0-2MM 100, available from http://www.honeyberryltd.co.uk/; fruits (fresh and frozen) from farm, e.g., in North Carolina; Chokeberry Powder 2.2 lbs, sold by NutriCargo, LLC (via Amazon.com); sloe, from Empresa de Bourbon SL, Navarra, Spain.

Dragon's Blood is a sap obtained from trees of Croton spp., which is used as antidiarrheal in the South America. The mechanism of action is similar to the mechanism of action of the dried blueberries, and it is the inhibition of the secretory ion channels in the intestine. The Dragon's Blood and its derivatives are highly (˜100%) efficient against calcium-dependent chloride channels in the intestine and are also efficient against other Cl⁻ channels. They will combat the secretory component of MVID diarrhea.

Dragon's Blood is known, and is commercially available, and in various forms. In liquid, raw form as “Dragon's Blood 30 ml/1.01 fl oz (100% Raw Croton Lechleri Sap)” from Alpha Naturals LLC (via Amazon.com); liquid, extract form as Dragon's Blood, liquid, extract, available as Sangre de Grado (aka Dragon's Blood) 1 oz” from Maca Magic store (via Amazon.com), and advertised as “100% Pure latex resin from Sangre de Grado tree trunks”; and Dragon's blood, powder, 6× concentrate, from “Swanson Superior Herbs, Swanson Health Products).

Preferred polyphenols include those shown to possess antiproliferative properties and/or inhibitory activity against ion and water transport proteins. It is a wide class of plant chemicals, which includes the larger proanthocyanidins, and also smaller derivatives of gallic acid. Hydrolysable (gallic acid), non-hydrolyzable (condensed) and phlorotannins are also contemplated. Tannins address the increased permeability and leakiness of the intestinal monolayer via the chemical stabilization of the surface proteins of the intestinal epithelium; they also have antisecretory and astringent properties (http://www.ncbi.nlm.nih.gov/pubmed/21748285). They will combat the secretory component of MVID diarrhea and the increased paracellular leakiness. Non-limiting examples of polyphenols include plant-derived and synthesized, OH— radical rich polyphenols, e.g. cyanidin, delphinidin, petunidin, pelargonidin, peonidin, malvidin, catechin, gallocatechin, epicatechin, epigallocatechin, quercetin, gallic acid, tannic acid, apigenin (penta-m-digalloyl glucose), tannin, Cesinex, Styphnasal N, Zyone as well as all polymers of thereof with degree of polymerization 1, 2 and above), glycosylated forms (i.e. glucopyranoside) and all other chemical forms and combinations of thereof; monomeric and polymeric, chemical forms (i.e. glycosides), and gallic acid derivatives, condensed tannins, apigenin (penta-m-digalloyl glucose), and particularly those shown to possess antiproliferative properties and/or activity against ion and water transport proteins. Some non-limiting examples of anthocyanidins include cyanidin, delphinidin, petunidin, pelargonidin, peonidin, malvidin, catechin, gallocatechin, epicatechin, epigallocatechin, quercetin, gallic acid, tannic acid, apigenin (penta-m-digalloyl glucose), tannin. Proanthocyanidins are polymers or aggregates or clusters of the anthocyandins: and in some embodiments, the proanthocyanidins may be hydrolyzed, to form the precursor anthocyandins.

Non-limiting examples of tannins include Tannic Acid (OS: JAN), CCRIS 571 (IS), Gallotannic acid (IS), Gallotannin (IS), NSC 656273 (IS), Tannin (IS), UNII-28F9E0DJY6 (IS), Tannic Acid (PH: BP 2015, JP XVI, USP 37), Tannic acid (PH: Ph. Eur. 8), Tanninum (PH: Ph. Eur. 8), Cesinex. Styphnasal N, Zyone.

The zinc salt, calcium salt, selenium salt, and/or bismuth salt is not particularly limited. In some embodiments, the salt is a sulfate salt, chloride salt, carboxylate salt, lactate salt, gluconate salt, stearate salt, palmitate salt, or a combination thereof.

One or more trace elements, such as zinc, calcium, selenium, bismuth and salts/derivatives of thereof, are present. Zinc has been shown to possess antidiarrheal properties and is recommended for the treatment of secretory diarrhea by world health organization (http://www.who.int/elena/titles/zinc_diarrhoea/en/). Calcium reduces permeability of the intestinal epithelium and paracellular leakiness. In addition, calcium facilitates appearance of microvilli on the surface of enterocytes. Selenium and bismuth have also been shown to possess antidiarrheal properties. Other examples include zinc sulfate, calcium lactate, and calcium gluconate.

In some embodiments, the salt is zinc sulfate.

The amounts of the retinoid, polyphenol, and salt are not particularly limited. The amounts for each can independently range from an Upper Tolerable Intake (UTI) to therapeutic range, which can if desired be obtained by clinical experiments or models, for example, using an animal model.

In some embodiments, the retinoid is present independently in an amount ranging from 0.01 milligrams to 50 milligrams. This range includes all values and subranges therebetween, including 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, and 50 milligrams, or any combination thereof.

In some embodiments, the polyphenol is present independently in an amount ranging from 0.05 milligrams to 10 grams. This range includes all values and subranges therebetween, including 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 milligrams, or any combination thereof.

In some embodiments, the zinc salt, calcium salt, selenium salt, bismuth salt, may be present, each independently, alone or combined, in an amount ranging from 0.1 milligrams to 225 milligrams. This range includes all values and subranges therebetween, including 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 210, and 225 milligrams, or any combination thereof.

In some embodiments, a daily dose for children, for example, may total 10 g/day for polyphenols, 225 mg/day for Zn salt and 10 mg of retinoid.

In some embodiments, the composition may further include one or more physiologically acceptable carrier, excipient, or diluent.

In one embodiment, the composition includes 300 micrograms of retinol palmitate, 4 milligrams of zinc sulfate, and 3.5 grams of Alisitol.

In some embodiments, the composition may be administered to a subject 1-5 times per day. This range includes all values and subranges therebetween, including 1, 2, 3, 4, and 5 times per day.

The subject may be suitably selected from a mammal, such as a human, although the composition may have applicability in veterinary applications, for example for domesticated animals, livestock, food animals, and the like.

In some embodiments, the the subject is a human of one or more of Turkish, Navajo, Saudi Arabian, Sicilian, or Korean descent.

The age of the subject is not particularly limited, and the composition may be administered to all ages.

The inventors consider that the diarrhea in MVID may result from the following abnormalities:
Reduced capacity of the intestinal epithelium to absorb critical electrolytes, such as Na⁺ and Cl⁻, and smaller nutrients, such as glucose and amino acids;
Reduced absorptive surface of the intestinal epithelium due to the villus and microvillus atrophy;
Unopposed secretion of ions (Cl⁻) and water through the crypts; and
Paracellular losses of fluid due to the increased permeability of epithelial barrier.

These abnormalities are believed to arise as a consequence of defective processes of cellular maturation and polarity in the absence of Myo5b. Myo5b is a molecular motor that normally delivers intracellular proteins to their proper locations, and its absence brings considerable discord in to the otherwise spatially and temporally coordinated events of the cell cycle. The result of the Myo5b deficiency is the intestine that is in the permanent secretory state, and severe diarrhea ensues. Intestinal cells, unlike other epithelial cells are unable to compensate for the deficiency of Myo5b transport because of lack of time dictated by the very short life span (less than 5 days) of the enterocytes. These are some of the shortest living cells in the body and they simply don't have a luxury of time to overcome the genetic defect and mature in to the normal absorptive villus cell, remaining permanently frozen in the crypt-like secretory state.

The hallmark morphological features of the disease are not uniformly observed in the epithelium (some intestinal cells display normal brush borders; inclusions are a rare phenomenon; some cells display normal expression and localization of the absorptive transporters), indicating availability of mechanisms that allow some cells to compensate for the deficiency of the Myo5b.

The inventors consider that these mechanisms are druggable targets in the etiologic treatment of MVID, and the diarrhea can be corrected with the proper selection and compounding of pharmacological treatments, in accordance with the teachings herein and the knowledge of one of ordinary skill.

The composition may suitably be used to treat one or both the symptoms and the pathogenesis of the disease and ameliorate the lethal diarrhea. In some embodiments, the symptoms are those of the secretory diarrhea. Without wishing to be bound by theory, it is believed that the mechanism includes the reduced expression of absorptive protein machinery, otherwise typical of fully differentiated enterocytes, in the milieu of unchanged or minimally affected secretory machinery due to the immature state of the cells of intestinal epithelium. Reduced expression of the absorptive proteins is a well-known cause of the secretory diarrhea in pediatric patients. It shifts the balance of absorption and secretion towards unopposed secretion leading to the intractable diarrhea. In some embodiments, targeted disease mechanisms may include:

Absent or non-functional Myo5b protein;

Reduced expression of NHE3, DRA. SGLT1 and any other protein relevant in the homeostasis of fluid, ions and nutrients;

Reduced function of the aforementioned proteins; and/or

Preserved or minimally reduced function of the secretory proteins.

The composition and method are applicable for treating, or ameliorating the symptoms, or reducing the pathogenesis of one or more of MVID, congenital chloride diarrhea, congenital sodium diarrhea, diarrhea associated with Cholera, traveler's diarrhea, infectious diarrhea, secretory diarrhea, diarrhea associated with enterocyte development abnormality, tufting enteropathy, bacterial diarrhea, viral diarrhea, diarrhea associated with rotavirus infection, or a combination thereof.

In some embodiments, the treatment is for MVID and/or congenital diarrhea (the difference is the presence of genetic defect in the pathogenesis of the disease). The ion channel underlying pathogenesis of Congenital Chloride Diarrhea is DRA; Ion channel underlying pathogenesis of Congenital Sodium Diarrhea is NHE3. Both Ion channels are in the pathogenesis of MVID and may be addressed by the composition and methods described herein.

In some embodiments, the composition may further include one or more of a chloride transport inhibitor, hormone, bradykinin inhibitor, enzyme, digestive enzyme, phorbol meristate acetate, proton pump inhibitor, loop diuretic, or a combination thereof.

One or more hormones and/or bioactive substances may be included. Since the diarrhea in MVID does not start until hours and days after birth, it is likely that maternal hormones of pregnancy protect baby intestine. Thus, they can be used in the treatment of the disease. Bradykinin is hypothesized to be a substance involved in the normal signaling and chemosensing in the intestine. Since bradykinin receptors are regulated by the Myosin V proteins, the disturbances in the BDK signaling resulting from the Myo5b deficiency must be addressed. Non-limiting examples include estrogens, progestines, human placental lactogen, hCG, prolactin, Insulin-like growth factor-1, glucose-dependent inotropic peptide, cholecystokinin, glucagon, neurotensin; Icatibant; ACE inhibitors: captopril, enalapril, lisinopril, fosinopril, etc.

The amount of hormones and/or bioactive substances is not particularly limiting. For example, if present, the amount of hormones may be 0.001-100 micrograms per dose. This range includes all values and subranges therebetween, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, and 100 micrograms, or any combination thereof, per dose.

Chloride transport inhibitors may be included, if desired. They work by inhibiting the Cl⁻ secretion in to the intestinal lumen. Many such inhibitors exist, including injectable forms and oral preparations. “CFTR inhibitors” are known, and refer to a compound or compounds that reduce the efficiency of ion transport by CFTR, particularly with respect to transport of chloride ions by CFTR. The CFTR inhibitors are not particularly limited and may be specific CFTR inhibitors, i.e., compounds that inhibit CFTR activity without significantly or adversely affecting activity of other ion transporters, e.g., other chloride transporters, potassium transporters; high-affinity CFTR inhibitors, e.g., have an affinity for CFTR of at least about one micromolar, usually about one to five micromolar, or the like. Drugs in this group will combat the secretory component of MVID diarrhea. Non-limiting examples include Dragon's Blood sap and bark extract and all its derivatives, including, but not limited to SP-303, Provir, SB-NSF, and Crofelemer. Crofelemer works by voltage-independently blocking two structurally unrelated chloride channels in the gut, namely the cystic fibrosis transmembrane conductance regulator (CFTR) with an in vitro maximum inhibition of about 60%, and the calcium-activated chloride channel anoctamin 1, with a maximum inhibition of over 90%. The substance is hardly, if at all, absorbed from the gut into the bloodstream, and is consequently excreted with the stool (from Wikipedia on Crofelemer). Further, CFTR stimulators, such as lumacaftor and ivacaftor, specifically modified to convert them in to the blockers of secretion and function of the CFTR channel (for example, by adding polar radicals) are contemplated.

In some embodiments, the chloride transport inhibitor is one other than Dragon's Blood. In other embodiments, the chloride transport inhibitor includes Dragon's Blood.

Also, CFTR inhibitors may include one or more of those developed by the group of A. Verkman and incorporated herein each independently by reference: U.S. patent and publication nos.: U.S. Pat. No. 7,888,332; 2009/0253799; 2008/0171793; 2015/0290175; U.S. Pat. Nos. 9,073,863; 9,062,073; 2014/0080821; 8,609,661; 8,552,067; 2013/0143765; 2012/0208822; 2012/0202876; and 2011/0105565.

The amount of chloride transport inhibitors is not particularly limiting. For example, if present, the amount of chloride transport inhibitors may be 0.01-1 grams per dose. This range includes all values and subranges therebetween, including 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 grams, or any combination thereof, per dose.

One or more loop diuretics may be included, if desired. They work by blocking the NKCC1/2 pump (http://www.ncbi.nlm.nih.gov/pubmed/11882915) and thus by inhibiting the entrance of the Cl⁻ ions in to the intestinal cells, limiting the number of ions available for secretion and thus reducing the concomitant water loss. (http://www.ncbi.nlm.nih.gov/pubmed/1005757). In addition, they may act via the GABA-dependent neurological pathways (http://www.ncbi.nlm.nih.gov/pmc/articles/PMC3396710/). They will counter the secretory component of MVID diarrhea. Non-limiting examples include bumethanide, furosemide, ethacrynic acid, and torsemide.

The amount of loop diuretics is not particularly limiting. For example, if present, the amount of loop diuretics may be 0.1-100 micrograms per dose. This range includes all values and subranges therebetween, including 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, and 100 micrograms, or any combination thereof, per dose.

One or more microtubule inhibitors may be included, if desired. They were shown to reduce the CFTR function in the electrophysiological experiments. They will combat the secretory component of MVID diarrhea. Non-limiting examples include cabazitaxel, colcemid, colchicine, cryptophycin, demeclocycline, docetaxel, nocodazole, paclitaxel, taccalonolide, taxane, vinblastine, and vincristine.

The amount of microtubule inhibitor is not particularly limiting. For example, if present, the amount of microtubule inhibitor may be 0.1-5 milligrams per dose. This range includes all values and subranges therebetween, including 0.1, 0.2, 0.3, 0.4, 0.5, 0.6, 0.7, 0.8, 0.9, 1, 2, 3, 4, and 5 milligrams, or any combination thereof, per dose.

One or more of phorbol meristate acetate (PMA) and its variants may be included, if desired. PMA is reported to possess antiproliverative properties. They will speed up the maturation of cells and recover the lost absorption of Na⁺, Cl⁻, glucose and amino acids via induction of the biosynthesis of the absorptive protein machinery.

The amount of phorbol meristate acetate and/or its variants is not particularly limiting. For example, if present, the amount may be 0.001-100 milligrams per dose. This range includes all values and subranges therebetween, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, and 100 milligrams, or any combination thereof, per dose.

One or more proton pump inhibitors may be included, if desired. By blocking the hydrochloric acid secretion in the stomach, they will reduce the Cl⁻ inflow from the stomach, thus reducing the osmolarity of the intestinal content. This will help to reduce the diarrhea via the anti-osmotic effect and reduction in the paracellular water losses. They can be used continuously for very long periods of time (more then 10 years) in children: http://www.ncbi.nlm.nih.gov/pubmed/17307542. Has been used in the treatment of congenital Cl⁻ diarrhea: http://www.ncbi.nlm.nih.gov/pubmed/21127979, http://www.ncbi.nlm.nih.gov/pubmed/8988888. Non-limiting examples include esomeprazole, lansoprazole, omeprazole, pantoprazole, rabeprazole, etc.

The amount of proton pump inhibitors is not particularly limiting. For example, if present, the amount may be 0.001-0.1 gram per dose. This range includes all values and subranges therebetween, including 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 grams, or any combination thereof, per dose.

One or more digestive enzymes may be included, if desired. Malabsorption is a likely consequence of the functional prematurity of the intestinal epithelial lining, because both digestion and absorption are the functions of mature villus enterocytes. From here, the use of slow- and controlled release intestinal enzyme preparations will be beneficial. It will provide a functional relief for the intestinal lining and ensure adequate digestion in the individuals with MVID. Enzyme preps containing the enteric enzymes (at least the enterokinase to activate trypsin and chymotrypsin) must be preferred over the pancreatic enzymes due to the predominant deficiency of the enteric brush border enzymes. Non-limiting examples include Pancrelipase, Creon, Mezim-Forte, Festal and other digestive enzymes, preferably with intestinal enzyme preparations.

Digestive enzymes: 100-250000 units per dose

The amount of digestive enzymes is not particularly limiting. For example, if present, the amount may be 100-250000 units per dose. This range includes all values and subranges therebetween, including 100, 200, 300, 400, 500, 600, 700, 800, 900, 1,000, 2,000, 3,000, 4,000, 5.000, 6.000, 7,000, 8,000, 9,000, 10,000, 20,000, 30,000, 40,000, 50,000, 60,000, 70,000, 80,000, 90,000, 100,000, 200,000, 210,000, 220,000, 230,000, 240,000, and 250,000 units, or any combination thereof, per dose.

One or more solutions may be included, if desired. After the diarrhea is controlled and per os feeding regiment is considered, the electrolyte solutions will help to provide a balanced composition of electrolytes. Non-limiting examples include electrolytic solutions, rehydration solutions, nutritional solutions, and the like. Non-limiting examples of rehydration solutions include oral rehydrations salts as defined by World Health Organization regulation # WHO/FCH/CAH/06.1 (http://apps.who.int/iris/bitstream/10665/69227/1/WHO_FCH_CAH_06.1.pdf?ua=1&ua=1), Pedialyte, and the like.

The amount of solution is not particularly limited. If present, the amount may range from 0.1-85% by weight of the composition. This range includes all values and subranges therebetween, including 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, and 85%, or any combination thereof, by weight of the composition.

Table 1, below, sets out some embodiments where numbers correspond to the codes and different classes and options of drugs used, or usable in some the compositions, from which in the formulas below, the combinations of numbers separated by the dot correspond to the combinations of ingredients.

TABLE 1

| | | from | from | from | from |
|---|---|---|---|---|---|
| Compound | Code | 0 y.o. | 1 y.o. | 3 y.o. | 12 y.o. |
| Renoids | 10 | + | + | + | + |
| Polyphenol | 20 | + | + | + | + |
| Trace elements | 30 | + | + | + | + |
| Hormones and | 100 | + | + | + | + |
| bioactive substances/ | | | | | |
| inhibitors of thereof | | | | | |
| Chloride transport | 200 | ? | ? | ? | + |
| inhibitors | | | | | |
| Loop diuretics | 300 | + | + | + | + |
| MT inhibitors | 400 | ? | + | + | + |
| PMA | 500 | ? | ? | ? | ? |
| PPI | 600 | + | + | + | + |
| Digestive enzymes | 700 | + | + | + | + |
| Oral rehydration | 800 | + | + | + | + |
| solutions | | | | | |

For convenience, the composition is sometimes generically referred to herein as “Shylicine.” Using the codes from Table 1 above, some non-limiting examples of Shylicine include: Shylicine 10.20.30; Shylicine 10.20.30.100; Shylicine 10.20.30.200; Shylicine 10.20.30.300; Shylicine 10.20.30.400; Shylicine 10.20.30.500; Shylicine 10.20.30.600; Shylicine 10.20.30.700; Shylicine 10.20.30.800; Shylicine 10.20.30.100.200.

Any further combinations of the above, i.e. 10.20.100, 10.100.700.800, 100.300.800 etc., up to 10.20.30.100.200.300.400.500.600.700.800, where numbers correspond to the codes from the Table 1, and combinations of numbers separated by the dot correspond to the combinations of the different classes of drugs used in the formulation. For all treatment options groups, codes may be expanded to accommodate for any subgroup. For instance, code 300 indicates a loop diuretic class of medications, and within the class, individual drugs will be assigned numerical identifiers, such as 301, bumetanide; 302, furosemide; and so on.

Formulation Considerations in Different Age Groups:

The age of the patient is not particularly limited, and may suitably range from newborn to adult. This range includes all values and subranges therebetween, including >0, 1, 2, 3, 4, 5, 6, 7, 8, 9, 10, 11, and 12 months, 1, 2, 3, 4, 5, 6, 7, 8, 9, 10, 11, 12, 13, 14, 15, 16, 17, and 18 years, and over 18 years.

Indications by Age:

Pediatric age groups: 0-6 month of age, 7-12 month of age, 1-3 years of age, 4-8 years of age 9-13 years of age and 14-18 years of age. Adults: over 18 years of age.

Neonatal formulations (0 m.o.-1 y.o.).

Key considerations: the digestive system is not mature yet; the baby cannot have solid foods; there are problems with swallowing pills; the voluntary control over swallowing is minimal. Thus, liquids seem to be the best drug delivery method in this age group. Initially, in the face of severe dehydration, formulations could be delivered through the nasogastric tube. As the baby recovers and starts tolerating oral intake, oral formulations in mother's milk, baby formula, cow/goat milk and oral rehydration solutions may take over. Intestinal sustained-release formulations are questionable in the face of severe diarrhea, which will limit the intestinal dwelling time of the drugs administered. Injectable drugs are a viable option, and the long acting slow-release depot injectable preparations are superior over the short-acting preparations.

Pediatric formulations (1-10 y.o.). A key distinction of this age group is the ability to take solid food and an increasingly better control over swallowing. Thus, the delivery options for formulations in this age group are liquids, pills and injectable forms. Liquids: in addition to the options described in the “neonatal formulations” section, a syrup form can be added to the line-up of oral formulations. Tableted or capsulated preparations may be made available for the advanced children able to swallow tablets. Dosage in the formulations must be corrected accordingly.

Adolescent and Adult Formulations:

Pills and capsules become the mainstream formulation in this group, f.b. injectables in the depot form. Daily or more frequent injections are a burden, much like insulin injections in the diabetics; so all short-acting preparations must be formulated for P/O administration and not for injections. In this age group the necessity for the liquid formulations is minimal. Composition and dosage of the formulations must be corrected accordingly.

Non-limiting examples of formulations, grouped by age, appear in Table 2, below.

TABLE 2

| Age group | Vit. A (retinol), ug/day | Zn, mg/day | Alisitol, g/day |
|---|---|---|---|
| 0-6 m.o. | 600 | 4 | 5 |
| 7-12 m.o. | 600 | 5 | 5 |
| 1-3 y.o. | 600 | 7 | 10 |
| 4-8 y.o. | 900 | 12 | 10 |
| 9-13 y.o. | 1700 | 23 | 10 |

In one embodiment, the composition includes 300 micrograms of Retinol palmitate, 4 milligrams of Zinc sulfate and 3.5 grams of Alisitol (Vaccinium cyanococcus spp. (fruit or dried powder of thereof)) per dose, administered 3 times a day.

In another embodiment, the composition includes 300 micrograms of Retinol palmitate, 4 milligrams of Zinc sulfate and 3.5 grams of Alisitol (Aronia melanocarpa spp. (fruit or dried powder of thereof)) per dose, administered 3 times a day.

In another embodiment, the composition includes 300 micrograms of Retinol palmitate, 4 milligrams of Zinc sulfate and 3.5 grams of Alisitol (Prunus spinose spp. (fruit or dried powder of thereof)) per dose, administered 3 times a day.

In another embodiment, the composition includes 300 micrograms of Retinol palmitate, 4 milligrams of Zinc sulfate and 3.5 grams of Alisitol per dose, administered 3 times a day.

In another embodiment, the composition may be in liquid form made of purified water, glycerol, maple syrup, malt extract and xanathan gum, containing the following active substances: Retinol acetate; Zn sulfate; Alisitol powder.

In another embodiment, the composition may be in liquid form made of purified water, glycerol, maple syrup, malt extract and xanathan gum, containing the following active substances: Retinol acetate; Zn sulfate; Alisitol liquid extract (water-based, distilled alcohol-based and glycerol-based)

In another embodiment, the composition may be in solid form made of water, gelatin, erythritol, glucose, sugar, containing the following active substances: Retinol acetate; Zn sulfate; Alisitol powder

In another embodiment, the composition may be in solid form made of water, gelatin, erythritol, glucose, sugar, containing the following active substances: Retinol acetate; Zn sulfate; Alisitol liquid extract (water-based, distilled alcohol-based and glycerol-based)

Another embodiment provides a composition, which includes the composition and a physiologically acceptable carrier.

Another embodiment provides a method, which includes administering the composition to a subject in need thereof, to treat said subject.

In some embodiments, the form of the composition is not particularly limiting.

In some embodiments, a composition is provided, which includes one or more of the compositions and optionally a physiologically acceptable carrier.

In some embodiments, a method is provided, which includes administering the composition to a subject in need thereof, to treat the subject.

In some embodiments the compositions and methods are suitable for the treatment in humans (either or both of immunocompetent and immunocompromised) and animals of MVID, secretory diarrhea, and the like described herein.

In some embodiments the compositions and methods are suitable for the treatment in humans (both immunocompetent and immunocompromised) and animals of MVID, secretory diarrhea, and the like described herein.

In some embodiments, the subject is known or suspected to need treatment for one or more maladies related to MVID, secretory diarrhea, and the like described herein.

In some embodiments the compositions and methods are suitable for use or treatment alone or in combination with one or more additional treatments, for example either concurrently or with delayed administration. For example, in etiologic treatment, such as intestine-specific and/or whole organism gene therapy.

Gene therapy is a novel and very promising method of treatment of the genetic diseases. Several prominent peculiarities make MVID an ideal target for gene therapy. The anatomical and histological structure of the intestine favors gene therapy, because the progenitor cells giving rise to the entire intestinal epithelial lining (the site affected by the MVID) are located in the intestinal crypts and thus are exposed to the intestinal luminal content. As known, gastrointestinal tract is a tube that connects oral and anal openings, keeping aggressive intestinal content outside the body and isolating the inside of the body from intestinal luminal content. This creates a unique opportunity to target epithelial cells of the intestinal lining with extraneous genetic material, which will always remain situated outside the body (in the intestinal lumen), tremendously reducing the chance of developing complications, side effects and adverse reactions.

MVID is a lethal genetic disorder resulting from mutations in Myo5b; thus, reintroducing the normal genetic template (example can be found here https://www.ncbi.nlm.nih.gov/gene/4645) for MYO5b gene, fragments of the gene, a cDNA or mRNA, and/or other fragments of the genetic material relevant for the treatment of MVID which will restore the lost Myosin 5b protein back to its functional state. Options for reintroduction are lentiviral, retroviral or adenoviral vectors that are modified for the delivery of human genetic material, (such as Gendicine™ http://www.nature.com/nbt/joumal/v22/n1/full/nbt0104-3.html). Other methods of genetic material delivery can also be used, such as the injection of naked DNA, electroporation, the gene gun, sonoporation, magnetofection, the use of oligonucleotides, lipoplexes, dedrimers and inorganic nanoparticles (from Wikipedia: https://en.wikipedia.org/wiki/Gene_therapy).

MVID is the intestine-specific disease, reducing the necessity for whole-body correction of the genetic defect and opening the possibility of organ-specific corrections (the intestine). In turn, it means that the viral vector carrying Myosin Vb genetic material can be delivered specifically to the site of action (small and large intestines) by using encapsulation, microencapsulation and time-released preparation techniques. This will help to ensure that other parts of digestive tract and the body in whole, such as mouth, pharynx, larynx, esophagus and stomach and other organs and tissues remain unaffected, minimizing the possibility of side effects.

The short lifespan of the enterocytes (3-5 days) means that the cells are rapidly shedding and renewing; after 5 days no cells that were directly exposed to the viral vector will remain in the intestinal lining, with the exception of the intestinal progenitor cells. This will help to minimize the adverse reactions on one hand, but on the other, it may necessitate several rounds of treatment with the viral vectors to increase its efficiency.

The genetic nature and severity of the MVID, anatomical and physiological features of the intestine and isolation of the intestinal environment make it an ideal platform for the gene therapy.

In some embodiments, the subject is mammalian, human, livestock, cow, horse, pig, or the like.

In some embodiments, the composition can be administered to a human patient by itself or in pharmaceutical compositions where it may be mixed with suitable carriers or excipients at doses to treat or ameliorate various conditions characterized by diarrhea. A therapeutically effective dose may refer to that amount of the composition sufficient to inhibit the diarrhea, it being understood that such inhibition may occur at different concentrations such that a person skilled in the art could determine the required dosage of composition to inhibit the target diarrhea. Therapeutically effective doses may be administered alone or as adjunctive therapy in combination with other treatments. Some examples of techniques for the formulation and administration of the compositions may be found in Remington's Pharmaceutical Sciences, 18th Edition, A. R. Gennaro, Ed., Mack Publishing Co., Easton, Pa. (1990).

Liquid and solid forms and/or extracts can be further modified by addition of the excipients.

Liquid and solid forms and/or extracts can be further modified by addition of the following excipients: sodium benzoate, polyethylene glycol, propylene glycol, hydroxyethyl cellulose, xanthan gum, sucralose, natural and artificial colorants.

Suitable routes of administration which are not intended to be limiting may include, for example, oral, intravenous, inhaled, intra-peritoneal, rectal, transmucosal, buccal, intra-vaginal, intestinal, topical, intradermal, parenteral delivery, intramuscular, subcutaneous, intramedullary injection, intrathecal, direct intraventricular, intravenous, intraperitoneal, intranasal, intraocular, peritoneal, pleural, and optionally in a depot or sustained release formulation. Furthermore, one may administer the composition in a targeted drug delivery system, for example in a liposome.

The compositions may be administered systemically (e.g. oral, intravenous, inhaled, intra-peritoneal, rectal) or locally (e.g., topical, intradermal, intrathecal, peritoneal, pleural, intraocular, intra-vesicular, intra-vaginal, or delivered specifically to the infection site).

The pharmaceutical compositions may be manufactured in a manner that is itself known, e.g., by means of conventional mixing, dissolving, dragee-making, levitating, emulsifying, encapsulating, entrapping, or lyophilizing processes. The pharmaceutical compositions thus may be formulated in conventional manner using one or more physiologically acceptable carriers comprising excipients and auxiliaries that facilitate processing of the active compounds into preparations, which can be used pharmaceutically. Proper formulation may be dependent upon the route of administration chosen.

Any combination of one or more the present compounds, salts thereof, resonance forms thereof, prodrugs, metabolites, isotopically-labeled compounds, tautomers, isomers, and/or atropisomers is possible in the composition.

For injection, the compositions may be formulated in aqueous solutions, preferably in physiologically compatible buffers, such as Hank's solution, Ringer's solution, or physiological saline buffer. For transmucosal administration, penetrants appropriate to the barrier to be permeated are used in the formulation. Such penetrants are known in the art.

For oral administration, the compositions can be formulated readily by combining the active compounds with pharmaceutically acceptable carriers well known to those in the art. Such carriers enable the compositions to be formulated as tablets, pills, dragees, capsules, liquids, gels, syrups, slurries, suspensions and the like, for oral ingestion by a patient to be treated. Pharmaceutical preparations for oral use can be obtained by combining the composition with a solid excipient, optionally grinding the resulting mixture, and processing the mixture of granules, after adding suitable auxiliaries, if desired, to obtain tablets or dragee cores. Suitable excipients include but are not limited to fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

The compositions may be formulated for parenteral administration by injection, e.g., by bolus injection or continuous infusion. Formulations for injection may be presented in unit dosage form, e.g., in ampoules or in multi-dose containers, with an added preservative, for example, sodium benzoate, sodium citrate, polypropylene glycol, and the like. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as polyionic block (co)polymer, sodium carboxymethyl cellulose, sorbitol, or dextran. Optionally, the suspension may also contain suitable stabilizers or agents which increase the solubility of the compounds.

Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

The compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid- or gel-phase carriers or excipients. Examples of such carriers or excipients include but are not limited to calcium carbonate, calcium phosphate, various sugars, starches, cellulose derivatives, gelatin, and polymers such as polyethylene glycols.

In some embodiments, the compounds may be provided as salts with pharmaceutically compatible counterions. Pharmaceutically compatible salts may be formed with many acids, including but not limited to hydrochloric, sulfuric, acetic, lactic, tartaric, malic, succinic, etc.; or bases. Non-limiting examples of pharmaceutically acceptable salts include sodium, potassium, lithium, calcium, magnesium, iron, zinc, hydrochloride, hydrobromide, hydroiodide, acetate, citrate, tartrate and maleate salts, and the like.

In some embodiments, other non-limiting drug delivery methods include:

Powder mixable with or dissolvable in mothers milk, other natural milk products (cow milk, goat milk) or artificial milk products such as baby formula;

Oral solution approved for use in the newborns (water-based, such as Oral Rehidration Solution. or oil-based, such as the vitamin D drops);

Oral Pills/tablets;

SM Pill™ packaging method (timed-release capsules with facilitated entry into the enterocytes http://www.sigmoidpharma.com/dynamicdata/ProprietaryTechnology.asp);

Nasogastric tube;

Injectable forms (subcutaneous, intramuscular, intravenous);

Injectable slow release depot forms;

Trans-dermal patches;

Intranasal drops; and

Enema.

Generally, pharmaceutical compositions contain the active compound in an effective amount to achieve their intended purpose. In one embodiment, a therapeutically effective amount means an amount effective to prevent or inhibit development or progression of a disease characterized by diarrhea in the subject being treated. Determination of the effective amounts is within the capability of those skilled in the art in light of the description provided herein.

The use of the word “a” or “an” when used in conjunction with the term “comprising” in the claims and/or the specification may mean “one,” but it is also consistent with the meaning of “one or more,” “at least one,” and “one or more than one.”

The term, “about” is used to indicate that a value includes the standard deviation of error.

The term, “or” means “and/or” unless explicitly indicated to refer to alternatives only or the alternatives are mutually exclusive, although the disclosure supports a definition that refers to only alternatives and “and/or.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other embodiments of the invention are discussed throughout this application. Any embodiment discussed with respect to one aspect applies to other embodiments as well and vice versa. Each embodiment described herein and any obvious variation thereof is understood to be applicable to all embodiments of the invention. Given the description herein, combined with the knowledge of one of ordinary skill in the art to which the invention pertains, any embodiment described herein can be easily accomplished and/or further implemented with respect to any use, method, composition, kit, obvious variant thereof, or any combination thereof.

### Examples

The Examples herein are provided for illustration, and are not intended to limiting unless otherwise specified.

1 gram of berry powder equals to 120 milligrams of extracted polyphenols. Volume of liquid extract to be used depends on the concentration of polyphenols in the extract.

Extraction Methods:

Water extraction: deionized water is used as a solvent. 0.33 g/ml of berry powder is mixed with water and incubated in the closed container for the periods of time from 30 minutes to 24 hours, at the range of temperatures from +4 to +100° C. After incubation, the remaining solid phase is separated from the liquid phase by means of centrifugation or filtration, and the concentration of polyphenols is measured in the supernatant/filtrate by means of spectrophotometry (by reading specific absorbance of the solution @ 750 nm in the presence of Folin-Ciocalteu reagent). Extraction can be repeated using the same powder and fresh water up to 6 times.

Alcohol extraction: distilled ethyl or methyl alcohol is used as a solvent. 40%, 50% or 70% alcohol in water has been used. 0.33 g/ml of berry powder is mixed with alcohol and incubated in the closed container for the periods of time from 30 minutes to 24 hours, at the range of temperatures from +4 to +100° C. After incubation, the remaining solid phase is separated from the liquid phase by means of centrifugation or filtration, and the concentration of polyphenols is measured in the supernatant/filtrate by means of spectrophotometry (by reading specific absorbance of the solution @ 750 nm in the presence of Folin-Ciocalteu reagent). Extraction can be repeated using the same powder and fresh solvent up to 6 times.

Acetone extraction: Pure acetone, or 50/50 V/V acetone in water has been used as solvent. 0.33 g/ml of berry powder is mixed with acetone and incubated in the closed container for the periods of time from 30 minutes to 24 hours, at the range of temperatures from +4 to +100° C. After incubation, the remaining solid phase is separated from the liquid phase by means of centrifugation or filtration, and the concentration of polyphenols is measured in the supernatant/filtrate by means of spectrophotometry (by reading specific absorbance of the solution @ 750 nm in the presence of Folin-Ciocalteu reagent). Extraction can be repeated using the same powder and fresh solvent up to 6 times.

Glycerol extraction: 100% glycerol or 70% glycerol in water or ethyl alcohol has been used as solvent. 0.33 g/ml of berry powder is mixed with glycerol and incubated in the closed container for the periods of time from 30 minutes to 24 hours, at the range of temperatures from +4 to +100° C. After incubation, the remaining solid phase is separated from the liquid phase by means of centrifugation or filtration, and the concentration of polyphenols is measured in the supernatant/filtrate by means of spectrophotometry (by reading specific absorbance of the solution @ 750 nm in the presence of Folin-Ciocalteu reagent). Extraction can be repeated using the same powder and fresh solvent up to 6 times.

Treatment example: A male patient born 2012, the fourth child of A1 Murrah origin, presented with a history of watery diarrhea since birth, failure to thrive and developmental delay. His diagnosis was originally Congenital Chloride Diarrhea (CCD) with raised level of chloride (>90 mmol/L) in stool in the absence of cystic fibrosis. Management included replacement of NaCl and KCl and correction of dehydration.

At age 11 months, the patient was referred by a pediatric gastroenterologist with the suspicion of Microvillus Inclusion Disease (MVID). The disease was genetically confirmed. The patient was then referred for management and has been seen every six months for the past three years. The parents refused, on religious grounds, to include the patient as a candidate for liver transplant. The patient has been on TPN since three days after birth.

At age 4 years, 6 months, the patient was given a 10 ml dose of Shylicine orally, together with a date honey syrup, to improve the taste. The dose was tolerated. TPN was not discontinued.

Shylicine was administered to the patient 10 ml three times per day. The Shylicine formulation contained 300 micrograms of retinol palmitate. 4 milligrams of zinc sulfate, and 3.5 grams of Alisitol per dose, administered 3 times a day. Day 2 showed no significant signs of cessation of watery stool. However, on Day 3, stool frequency abruptly went from 8 times per day to 5 times per day. On Day 4, stool frequency was recorded as twice per day. On Day 9, the patient stool output was regular and showed signs of a thickening paste. TPN was stopped on Day 10 and has never been reinstated since.

Since from about Day 10, the child now eats regularly, has gained 6.37 Kg and passes solid (thick paste consistency) stool.

At age 4 years, 8 months, or about two months after Shylicine treatment was commenced, the patient's laboratory tests showed normal erythrocyte sedimentation rate, immunoglobulin levels, serum urea, creatinine, alkaline phosphatase, and albumin. Faecal quantitative fat, lactose hydrogen breath test, α₁ antitrypsin, and α₁ antitrypsin clearance were normal. There was no metabolic alkalosis and aldosterone (120 pmol/l) and renin (1.10 ng/l/s) levels were within the normal range. Plasma angiotensin converting enzyme levels were slightly elevated (80 U/l, normal adult range 0-75 U/l). Serum Na, Cl, Ca, Mg. and phosphate concentrations were within the normal range but he was slightly hypokalaemic (serum potassium 2.7-3.4 mmol/l). Urine analysis was normal. Faecal CI concentration was 152 mmol/l, Na 88 mmol/l, and K 38 mmol/l, fulfilling the two diagnostic criteria of CLD (faecal chloride content >90 mmol/l and faecal cationic gap F-Na⁺+K⁺<Cl⁻).

Schedule for pre-clinical trials for Shylicine: MVID mouse models will be used for obtaining pre-clinical validation of dosage, safety and efficacy.

Mouse models are significantly better then cellular models from the standpoint of faithful syndrome reproduction. The Myo5b knockout (KO) pups present with diarrhea and a very short life span of less than 12 hours and thus constitute a valid model of the disease. Relevant animal models of the disease did not exist until the year of 2015. The world's first mouse model of MVID was generated in the summer of 2015 (Carton-Garcia et al., 2015) and validated in March, 2016 (Weis et al., 2016).

Using the Spanish Myo5b mouse model (Carton-Garcia et al., 2015) the efficacy of Shylicine will be evaluated by defined protocol. The mouse model will also be used to confirm dose parameters.

Dose of Shylicine will be recalculated for the animals by using formula (Body surface area=K*mass^{0.667}, where K is the coefficient varying from 8.475 for the mouse pups to 9.8 for adult animals and mass is expressed in grams).

Increased life span is the most critical parameter of the study. We expect to see dose-dependent prolongation of the lifespan in response to the treatment.

Stool frequency, stool volume and a change from the liquid to solid stool will be monitored. We expect dose-dependent reduction in stool volume and frequency, and solidification of the stool.

In addition, blood pH normalization, blood glucose and electrolyte normalization are the systemic parameters that are expected to improve with Shylicine treatment. They will be monitored in mouse plasma following the treatment with Shylicine.

Intestinal morphology will be assessed following treatment with Shylicine. Increased length of intestinal villi and increased length and density of intestinal microvilli are the expected outcomes of the treatment.

Diarrhea is a syndrome that currently cannot be replicated in the cellular models. Thus, preclinical in vitro studies are planned to verify the details of the mechanism of action of Shylicine this will be done by examining changes in expression (RNA and protein levels) and localization of the critical absorptive machinery affected by MVID (i.e. DRA, NHE3, SGLT1). We will use the maturation-relevant cellular model of MVID (Kravtsov et al., 2016) for confirming the mechanism of action of Shylicine.

The entire contents of each reference, patent document, article, hyperlink, and the like described herein are hereby independently incorporated by reference.

What is claimed is:

1. A composition, comprising:
d. a retinoid;
e. a polyphenol; and
f. a zinc salt, calcium salt, selenium salt, bismuth salt, or a combination thereof.

2. The composition of claim 1, which is suitable for administration to a subject having or suspecting to have one or more of Microvillus Inclusion Disease, congenital chloride diarrhea, congenital sodium diarrhea, diarrhea associated with Cholera, traveler's diarrhea, infectious diarrhea, secretory diarrhea, diarrhea associated with enterocyte development abnormality, tufting enteropathy, bacterial diarrhea, viral diarrhea, diarrhea associated with rotavirus infection, or a combination thereof.

3. The composition of claim 1, wherein the retinoid is one or more of carotenoid, vitamin A, oral vitamin A, systemic Vitamin A, retinol, retinal, tretinoin, retinoic acid, isotretinoin, alitretinoin, etretinate, acitretin, tazarotene, bexarotene, adapalene and pyranon-derived retinoids, retinol palmitate, retinol acetate, Seletinoid G, 3,7-dimethyl-9-(2,6,6,trimethyl-1-cyclohexen-1-yl)-2,4,6,8-natetraen-1-ol, 3-dehydroretinol, Accutane®, acitretin, adapalene, alitretinoin, all-trans retinoic acid, Altinac®, Amnesteem®, antixerophthalmic vitamin, Aquasol A®, Avita®, axerophtholum, beta-carotene, beta-carotene oleovitamin A, bexarotene, Differin®, etretinate, isotretinoin, Palmitate-A®, Renova®, Retin-A®, Retin-A Micro®, retinaldehyde (RAL), retinyl acetate, retinyl N-formyl aspartamate, retinyl palmitate, retinol, Solatene®, Soriatane®, SourceCF®, Targretin®, tazarotene, Tazorac®, Tegison®, topical retinoids, tretinoin, Vesabiod®, Vesanoid®, Vitamax®, vitamin A USP, vitamin A1, vitamina A, vitaminum A, or a combination thereof.

4. The composition of claim 1, wherein the retinoid is a carotenoid.

5. The composition of claim 1, wherein the retinoid is one or more of vitamin A, retinol, retinal, tretinoin, retinoic acid, isotretinoin, alitretinoin, etretinate, acitretin, tazarotene, bexarotene, adapalene and pyranon-derived retinoids, retinol palmitate, retinol acetate, or a combination thereof.

6. The composition of claim 1, wherein the retinoid is vitamin A, retinol palmitate, retinol acetate, or a combination thereof.

7. The composition of claim 1, wherein the polyphenol is a synthetic or naturally occurring polyphenol rich in —OH groups and having antiproliferative properties and/or inhibitory activity against ion and water transport proteins.

8. The composition of claim 1, wherein the polyphenol is one or more of plant-derived, botanical, anthocyanoside, anthocyanidin, proanthocyanidin, tannin, or a combination thereof.

9. The composition of claim 1, wherein the polyphenol is one or more of Alisitol, crofelemer, blueberry (Vaccinium cyanococcus spp), bilberry, sloe (Prunus Spinosa spp.), chokeberry (Aronia melanocarpa spp.), grape (Cabbernet sauvignon spp.), grape pomace, black rose, blackcurrant (Ribes nigrum spp.), pecarin, Dragon's Blood (Croton spp.) dried form thereof, seed form thereof, juice thereof, powder form thereof, liquid form thereof, extract thereof, or combination thereof.

10. The composition of claim 1, wherein the polyphenol is one or more of gallic acid, hydrolysable gallic acid, non-hydrolyzable gallic acid, condensed gallic acid, phlorotannin, cyanidin, delphinidin, petunidin, pelargonidin, peonidin, malvidin, catechin, gallocatechin, epicatechin, epigallocatechin, quercetin, tannic acid, apigenin, penta-m-digalloyl glucose, tannin, condensed tannin, gallotannic acid, gallotannin, tanninium, Cesinex, Styphnasal N, Zyone, monomeric form thereof, polymer thereof having degree of polymerization of 1, 2 or more, glycosylated form thereof, salt thereof, ester thereof, or combination thereof.

11. The composition of claim 1, wherein the salt is a sulfate salt, chloride salt, carboxylate salt, lactate salt, gluconate salt, stearate salt, palmitate salt, or a combination thereof.

12. The composition of claim 1, wherein the salt is zinc sulfate.

13. The composition of claim 1, wherein
a. the retinoid is present in an amount ranging from 0.01 milligrams to 50 milligrams;
b. the polyphenol is present in an amount ranging from 0.05 milligrams to 10 grams; and
c. the zinc salt, calcium salt, selenium salt, bismuth salt, or a combination thereof is present in an amount ranging from 0.1 milligrams to 225 milligrams.

14. The composition of claim 1, further comprising one or more of a chloride transport inhibitor, hormone, bradykinin inhibitor, enzyme, digestive enzyme, phorbol meristate acetate, proton pump inhibitor, loop diuretic, or a combination thereof.

15. The composition of claim 1, further comprising one or more physiologically acceptable carrier, excipient, or diluent.

16. The composition of claim 1, comprising 300 micrograms of retinol palmitate, 4 milligrams of zinc sulfate, and 3.5 grams of Alisitol.

17. A method for treating a subject having or suspecting to have one or more of MVID, congenital chloride diarrhea, congenital sodium diarrhea, diarrhea associated with Cholera, traveler's diarrhea, infectious diarrhea, secretory diarrhea, diarrhea associated with enterocyte development abnormality, tufting enteropathy, bacterial diarrhea, viral diarrhea, diarrhea associated with rotavirus infection, or a combination thereof, comprising administering to said subject the composition of claim 1.

18. The method of claim 17, further comprising administering said composition to said subject 1-5 times per day.

19. The method of claim 17, wherein the subject is a human of 18 years old or younger.

20. The method of claim 17, wherein the subject is a human of one or more of Turkish, Navajo, Saudi Arabian, Sicilian, or Korean descent.
